(19) United States
(12) Patent Application Publication
(10) Pub. No.: US 2023/0075083 A1
(43) Pub. Date: Mar. 9, 2023

(54) ARTICLE IN THE FORM OF EDIBLE SHEET

(71) Applicant: TECOZYME INC., Seoul (KR)

(72) Inventor: Yoon NAM, Seoul (KR)

(73) Assignee: TECOZYME INC., Seoul (KR)

(21) Appl. No.: 17/596,646

(22) PCT Filed: May 25, 2021
(86) PCT No.: PCT/KR2021/006495

§ 371 Date: Dec. 15, 2021

(30) Foreign Application Priority Data
Jul. 24, 2020 (KR) 10-2020-0092654

(51) Int. Cl. A23L 27/00; A23L 33/125; A23L 29/262; A23L 33/16

(52) U.S. Cl. CPC A23L 27/79; A23L 33/125; A23L 29/262; A23L 33/16

(57) ABSTRACT

The present disclosure relates to an article in the form of an edible sheet. The edible sheet of the present disclosure can be processed into a thin form such as a sheet, has excellent adhesiveness to the mucous membrane in the oral cavity, has excellent durability, and is decomposed at an appropriate rate by moisture in the oral cavity, and can be used for various purposes.

### CROSS REFERENCE TO RELATED APPLICATIONS

This application is a 371 of PCT/KR2021/006495, filed May 25, 2021 which claims the benefit of Korean Patent Application No. 10-2020-0092654, filed Jul. 24, 2020, the contents of each of which are incorporated herein by reference.

### FIELD OF THE INVENTION

The present disclosure relates to an article in the form of an edible sheet.

### BACKGROUND OF THE INVENTION

Hyaluronic acid is a natural substance synthesized in the human body, and particularly, it is known that it is abundant in the skin. It is known to constitute the dermis together with an elastin protein tissue or a collagen protein tissue in the skin in a gel-phase under general conditions.

Since such hyaluronic acid contains many hydroxy groups in terms of molecular structure, it has very high hydrophilicity, water retention, and water-bearing properties, and thus it is known to have a moisturizing or absorbent action that supplies moisture to the skin from within the skin.

In addition, as it is a material that is bio-synthesized in the human body, it is known to be very safe for human use, and is widely used as an additive in cosmetics, pharmaceuticals, or eye drops.

Recently, a lot of health functional foods and general foods in particular for the purpose of directly ingesting hyaluronic acid itself have been released, and many applications are being attempted in the form of hydrogels, freeze-dried foams, and the like.

However, such hyaluronic acid is a polysaccharide-type polymer material in which a portion of the hydroxy group is substituted with an amide bond. Since it exists as a unique gel phase under general conditions or conditions in the human body, it is very difficult to process it into a thin form such as a sheet.

### DESCRIPTION OF THE INVENTION

### Technical Problem

The present specification provides an edible sheet processed into a thin form such as a sheet including hyaluronic acid.

### Technical Solution

The present specification provides an edible sheet including hyaluronic acid and hypromellose.

The hyaluronic acid may include one or more of a cross-linked hyaluronic acid and a non-cross-linked hyaluronic acid.

In addition, the hypromellose may preferably have a methoxy substitution rate of about 10 to about 35%, or about 20 to about 25%, and a hydroxypropyl substitution rate of about 1 to about 15%, about 3 to about 15%, or about 5 to about 10%.

According to an embodiment of the invention, the edible sheet may preferably have a dry thickness of about 0.05 to about 1 mm, or about 0.1 to about 0.5 mm. In this connection, the drying may be performed by a drying method for about 18 to about 30 hours under conditions of about 25 to about 45° C., preferably for about 18 to about 30 hours at a temperature condition similar to human body temperature, or a rapid drying method at a high temperature of about 80 to about 120° C. for about 10 to about 60 minutes.

In addition, the edible sheet may include about 0.1 to about 20 parts by weight of a hyaluronic acid, based on 100 parts by weight of hypromellose. The lower limit of the content of a hyaluronic acid may be about 0.1 parts by weight or more, preferably about 1 part by weight or more, or about 5 parts by weight or more, and the upper limit of the content of a hyaluronic acid may be about 20 parts by weight or less, or about 16 parts by weight or less.

In another aspect, the edible sheet may include about 0.1 to about 18 parts by weight of a hyaluronic acid and about 82 to about 99.9 parts by weight of hypromellose, based on a total of 100 parts by weight of a hyaluronic acid and hypromellose, preferably about 1 to 15 parts by weight of a hyaluronic acid and 85 to 99 parts by weight of hypromellose, or about 4 to 14 parts by weight of a hyaluronic acid and 86 to 96 parts by weight of hypromellose.

In addition, the hyaluronic acid may preferably have a weight average molecular weight of about 10,000 to about 1,000,000 g/mol, about 10,000 to about 100,000 g/mol, or about 10,000 to about 50,000 g/mol.

In addition, the hypromellose may preferably have a weight average molecular weight of 100,000 to 1,000,000 g/mol or about 100,000 to about 500,000 g/mol.

According to another embodiment of the invention, the edible sheet may further include ions of one or more edible metals selected from the group consisting of zinc, copper, iron, nickel, manganese, chromium, calcium, magnesium, sodium, potassium and selenium, in addition to hyaluronic acid and hypromellose.

In this connection, the hyaluronic acid and the hypromellose may be in the form of a complex coordinated around the metal, in other words, in the form of a metal coordination compound.

In addition, in this connection, the ion of the metal, the ion of the metal, may be included in a ratio of 0.01 to 2 moles with respect to a total of 100 g of the hyaluronic acid and the hypromellose, preferably a ratio of 0.1 to 0.5 moles with respect to a total of 100 g of the hyaluronic acid and the hypromellose, or a ratio of 0.15 to 0.35 moles with respect to a total of 100 g of the hyaluronic acid and the hypromellose.

The edible sheet according to an embodiment of the present disclosure i) may have a form that additionally includes a material other than hyaluronic acid and hypromellose, or ii) does not contain a material other than hyaluronic acid and hypromellose, and may consist only of a hyaluronic acid and hypromellose.

Terms used herein are used to describe exemplary embodiments and are not intended to limit the scope of the present disclosure.

The terms of a singular form may include plural forms unless otherwise specified.

As used herein, the terms “comprising,” “including” or “having,” or the like are used to specify the presence of stated features, integers, steps, components, or a combination thereof, but do not preclude the presence or addition of one or more other features, integers, steps, components, or combinations thereof.

In addition, as used herein, each layer or element is referred to as being formed “on” or “over” respective layers or elements, which means that each layer or element may be formed directly on respective layers or elements or another layer or element may be additionally formed between respective layers or on a target material or substrate.

The present disclosure may be variously modified and have various types, and specific embodiments of the present disclosure will be descried below in detail with reference to the accompanying drawing. However, the present disclosure is not limited to the exemplary embodiments described herein, but all of the modifications, equivalents, and substitutions within the spirit and scope of the present disclosure are also included in the present disclosure.

Hereinafter, the present disclosure will be described in detail.

According to one aspect of the present disclosure, there is provided an edible sheet including hyaluronic acid and hypromellose.

The inventors of the present disclosure completed the present disclosure based on the fact that when only hyaluronic acid is used alone, due to its unique gel form, it is very difficult to process it in a film or sheet form, and even when processed in a thin film or sheet form, it is easily decomposed in the oral cavity when ingested, its form disintegrates immediately, whereas when hyaluronic acid and hypromellose are used together, it can be processed relatively easily into a thin film or sheet form, and even when ingested, its form is maintained to some extent in the oral cavity.

The hyaluronic acid may include one or more of cross-linked hyaluronic acid and non-cross-linked hyaluronic acid.

The cross-linked hyaluronic acid means that in non-cross-linked hyaluronic acid in the form of a polysaccharide linear polymer in which a portion of the hydroxy group is substituted with an amide bond, a crosslinking bond is formed between each linear polymer.

Such cross-linked hyaluronic acid has established in vivo safety as much as non-cross-linked hyaluronic acid, and due to its unique cross-linked structure, when cross-linked hyaluronic acid and non-cross-linked hyaluronic acid are used simultaneously, it is possible to control the rate of dissolution in the oral cavity when ingested.

Hypromellose is a substance in which a portion of the hydroxy group present in the cellulose polymer is substituted with a methoxy group or a hydroxypropoxy group, and is also called hydroxypropyl methyl cellulose.

Hypromellose is in the form of a pale yellow to white powder or granule, and when water is added thereto, it increases in volume and has a mucus form.

Hypromellose also has established safety for the human body to some extent, and due to the above properties or the influence of hydroxy groups present a lot in molecules, it is widely used as an emulsifier, thickener, suspension stabilizer, etc., and is also used as food coating ingredients.

For such hypromellose, the physical properties of the polymer may vary depending on the degree of substitution of a methoxy group or a hydroxypropoxy group in the hydroxy group originally present in the cellulose polymer. When considering compatibility with hyaluronic acid, processability when processing in a sheet form, rate of decomposition in the oral cavity when ingested, and degree of decomposition, it may be preferable to use those having a methoxy substitution rate of about 10 to about 35%, or about 20 to about 25%, and a hydroxypropyl substitution rate of about 3 to about 15%, or about 5 to about 10%.

As described above, both hyaluronic acid and hypromellose are polymers in which repeating units of monosaccharides in the form of hexagonal rings are long linked, and have very similar molecular structures and have excellent compatibility because there are many hydroxy groups in the molecule.

In the case of using hyaluronic acid and hypromellose together, according to the above-described technical principle, the disadvantages of using each of a hyaluronic acid and hypromellose alone can be effectively compensated. In particular, when processed into a thin form such as a sheet or film, its durability or adhesion degree attached to the mucous membrane in the oral cavity is greatly increased.

In other words, the sheet or food in a film form containing hyaluronic acid and hypromellose is not easily decomposed in the oral cavity when ingested, and when it is attached to the oral mucosa in the oral cavity, it is not easily detached from the attachment site.

In consideration of the above characteristics, the edible sheet according to an embodiment of the present disclosure may be used as a simple edible film, and may also be used as a patch attached to a specific site in the oral cavity.

When used for such a use as a patch, the film or sheet slowly disintegrates in the oral cavity by including pharmaceuticals of specific ingredients, sweeteners, various vitamins, and/or various functional substances having an anti-odor function in the film or sheet, and may be used for the purpose of maintaining the oral use uniformly for a specific period of time. Alternatively, by attaching the film or sheet to the mucous membrane in the oral cavity, it may be used for uniform absorption into the mucous membrane for a specific period of time.

In addition, in this connection, the above-described functional substances such as pharmaceutical ingredients and sweeteners are not contained in the edible sheet itself of the present disclosure, but are contained in a kind of patch bag made of the edible sheet, or may be made in the form of being additionally coated or added to the top of the surface of the edible sheet of the present disclosure.

According to an embodiment of the invention, the edible sheet may preferably have a dry thickness of about 0.05 to about 1 mm, or about 0.1 to about 0.5 mm. In this connection, the drying may be performed by a drying method for about 18 to about 30 hours under conditions of about 25 to about 45° C., preferably for about 18 to about 30 hours at a temperature condition similar to human body temperature, or a rapid drying method at a high temperature of about 80 to about 120° C. for about 10 to about 60 minutes.

When the dry thickness of the edible sheet is in the above range, it is possible to maintain adequate strength in the oral cavity when food is ingested, and while the shape is released over time, it may properly perform the role as a patch or film.

In addition, the edible sheet may include about 0.1 to about 20 parts by weight of a hyaluronic acid, based on 100 parts by weight of hypromellose. The lower limit of the content of a hyaluronic acid may be about 0.1 parts by weight or more, preferably about 1 part by weight or more, or about 5 parts by weight or more, and the upper limit of the content of a hyaluronic acid may be about 20 parts by weight or less, or about 16 parts by weight or less.

In another aspect, the edible sheet may include about 0.1 to about 18 parts by weight of a hyaluronic acid and about 82 to about 99.9 parts by weight of hypromellose, based on a total of 100 parts by weight of a hyaluronic acid and hypromellose, preferably about 1 to 15 parts by weight of a hyaluronic acid and 85 to 99 parts by weight of hypromellose, or about 4 to 14 parts by weight of a hyaluronic acid and 86 to 96 parts by weight of hypromellose.

In the edible sheet according to an embodiment of the present disclosure, when the relative content of a hyaluronic acid and hypromellose is in the above range, the mutual complementary effect of the above-described hyaluronic acid and hypromellose can be maximized. In particular, adhesiveness and durability to mucous membranes in the oral cavity can be greatly improved.

In particular, there is always a lot of moisture in the oral cavity by saliva, and stimulation may be constantly applied by the tongue, teeth, cheeks, etc. In this aspect, when the relative content of a hyaluronic acid is too much or too little, the adhesiveness of the edible sheet in the oral cavity is lowered, or the durability of the sheet is lowered.

In addition, the hyaluronic acid may preferably have a weight average molecular weight of about 10,000 to about 1,000,000 g/mol, about 10,000 to about 100,000 g/mol, or about 10,000 to about 50,000 g/mol.

In addition, the hypromellose may preferably have a weight average molecular weight of 100,000 to 1,000,000 g/mol or about 100,000 to about 500,000 g/mol.

When the weight average molecular weight of a hyaluronic acid and hypromellose is within the above range, the above-described interaction and complementary effects of a hyaluronic acid and hypromellose can be maximized. In particular, adhesiveness to mucous membranes in the oral cavity, durability, and degradability over time can be appropriately adjusted.

According to another embodiment of the invention, the edible sheet may further include ions of one or more edible metals selected from the group consisting of zinc, copper, iron, nickel, manganese, chromium, calcium, magnesium, sodium, potassium and selenium, in addition to hyaluronic acid and hypromellose.

The above-mentioned ions of metals are generally present in a polycation state. It is possible to further improve the adhesiveness and durability of the edible sheet in the oral cavity by electrostatic interaction with numerous hydroxy groups present in hyaluronic acid and hypromellose contained in the edible sheet of the present disclosure.

In this connection, the hyaluronic acid and the hypromellose may be in the form of a complex coordinated around the metal, that is, in the form of a metal coordination compound.

In addition, the above-mentioned metals are mineral components helpful to the human body, and when such edible metals are included, the edible sheet according to an embodiment of the present disclosure may be used as health supplements.

In addition, in this connection, the ion of the metal, the ion of the metal, may be included in a ratio of 0.01 to 2 moles with respect to a total of 100 g of the hyaluronic acid and the hypromellose, preferably a ratio of 0.1 to 0.5 moles with respect to a total of 100 g of the hyaluronic acid and the hypromellose, or a ratio of 0.15 to 0.35 moles with respect to a total of 100 g of the hyaluronic acid and the hypromellose.

However, the present disclosure is not necessarily limited to the above range, and the content of these metals may vary depending on the above-described electrostatic interaction and recommended daily intake for each metal.

When the content of metal ions is too small, the above-mentioned beneficial effect may not be implemented well. When the content of metal ions is too high, the amount of absorption of metal ions into the human body increases, resulting in side effects. In addition, the electrostatic interaction with numerous hydroxy groups present in hyaluronic acid and hypromellose becomes too large, so that the edible sheet according to an embodiment of the present disclosure is not properly decomposed in the oral cavity.

In another aspect, the edible sheet may include about 0.1 to about 18 parts by weight of a hyaluronic acid and about 82 to about 99.9 parts by weight of hypromellose, based on a total of 100 parts by weight of a hyaluronic acid and hypromellose, preferably about 1 to 15 parts by weight of a hyaluronic acid and 85 to 99 parts by weight of hypromellose, or about 4 to 14 parts by weight of a hyaluronic acid and 86 to 96 parts by weight of hypromellose.

In other words, the edible sheet according to an embodiment of the present disclosure i) may have a form that additionally includes a material other than hyaluronic acid and hypromellose, or ii) does not contain a material other than hyaluronic acid and hypromellose, and may consist only of a hyaluronic acid and hypromellose.

In the case of an edible sheet composed only of a hyaluronic acid and hypromellose, it may be used simply as a snack food. In addition, as described above, the edible sheet may be used as a form containing various additives or functional substances in a kind of patch bag made of an edible sheet, other various additives or functional materials may be used in the form of being additionally coated or added on the surface of the edible sheet.

Examples of such additives include natural or synthetic sweeteners such as xylitol, menthol, aspartame, sodium saccharin, sucralose, sorbitol, acesulfame potassium, neopharm, stevia, and polydextrose; medicinal ingredients of naturally-derived extracts such as green tea, black tea, ginseng, and red ginseng such as catechins, saponins, and ginsenosides; and additives for formulation such as glycerin.

The edible sheet of the present disclosure may be prepared by the following method.

First, hyaluronic acid and hypromellose are dissolved in an appropriate amount of purified water. In this case, the purified water is preferably used in an amount of about 2 to about 10 times the total weight of the hyaluronic acid and hypromellose. In addition, for uniform dispersion of a hyaluronic acid and hypromellose at this time, it may be preferable to maintain the temperature of the water at about 50° C. or more, about 70° C. or more, or about 80 or more, and about 100° C. or less. At this time, also for uniform dispersion of a hyaluronic acid and hypromellose, a stirrer or the like may be used.

When an additive other than a hyaluronic acid is used, a separate additive is added to the solution in which a hyaluronic acid and hypromellose are dispersed as described above, or an additive solution is prepared separately therefrom, and then is prepared in a two-component liquid form or a three-component liquid form.

In the case of preparing additive solution in a two-component liquid form or a three-component liquid form, the type of liquid forms or the solubility temperature may be set in consideration of the solubility characteristics of each additive.

For example, in the case of an additive having high solubility in water at room temperature, an aqueous solution at room temperature may be prepared. In the case of an additive that is not highly soluble in water at room temperature, a separate aqueous solution or dispersion may be prepared in the form of dissolving the additive therein by preparing water at a high temperature. In the case of dissolving the additive in water at a high temperature, it may be used in combination with the solution for dispersing the hyaluronic acid and hypromellose.

When prepared as a two-component liquid form or a three-component liquid form as described above, a uniform mixture may be prepared by mixing the separately prepared additive liquid with the dispersion of a hyaluronic acid and hypromellose as described above under stirring.

When this is stirred at room temperature, hyaluronic acid and hypromellose change from a sol phase to a gel phase, but it does not change to a complete gel phase, and may exist in a half gel in the form of a fluid with high viscosity.

Next, the process of aging this half-gel-form mixture is performed. The aging process is a process of properly removing bubbles and an appropriate amount of water present in the half-gel-form mixture, putting the half-gel-form mixture in an airtight container, and leaving it at room temperature for about 1 to about 3 days. While left in the airtight container, the bubbles in the half-gel-form mixture escape, and an appropriate amount of water evaporates and forms on the upper portion of the airtight container. In order to efficiently remove these bubbles and evaporated water, it is also possible to adopt a method configured to observe whether water droplets form on the upper portion of the airtight container, properly open the lid to remove the water droplets from the upper portion, and then close the lid again.

After this aging process, the half-gel-form mixture has a higher viscosity and is shaped closer to a gel.

Such a gel may be processed into a sheet-type formulation such as a patch or a film through a drying process.

In this processing process, a release film may be used.

Specifically, using a coating equipment such as a bar coater, a spin coater, or an applicator, the gel-form mixture is uniformly applied on the release film. In consideration of the thickness after drying, the application thickness may be applied to about 2 times to about 5 times, or about 2 times to about 4 times the dry thickness.

After uniform application is made, it is transferred to a dryer to proceed with the drying process.

The drying may be performed by a method of dying for about 18 to about 30 hours under conditions of about 25 to about 45° C., preferably a method of drying for about 18 to about 30 hours at a temperature condition similar to human body temperature, or a method of rapidly drying at a high temperature of about 80 to about 120° C. for about 10 to about 60 minutes.

Through the drying process as described above, a food in the form of a sheet having a dry thickness of about ½ to about ¼ of the coating thickness may be prepared.

Terms used herein are used to describe exemplary embodiments and are not intended to limit the scope of the present disclosure.

The terms of a singular form may include plural forms unless otherwise specified.

As used herein, the terms “comprising,” “including” or “having,” or the like are used to specify the presence of stated features, integers, steps, components, or a combination thereof, but do not preclude the presence or addition of one or more other features, integers, steps, components, or combinations thereof.

In addition, as used herein, each layer or element is referred to as being formed “on” or “over” respective layers or elements, which means that each layer or element may be formed directly on respective layers or elements or another layer or element may be additionally formed between respective layers or on a target material or substrate.

The present disclosure may be variously modified and have various types, and specific embodiments of the present disclosure will be descried below in detail with reference to the accompanying drawing. However, the present disclosure is not limited to the exemplary embodiments described herein, but all of the modifications, equivalents, and substitutions within the spirit and scope of the present disclosure are also included in the present disclosure.

Hereinafter, the present disclosure will be described in detail.

### Advantage of the Invention

The edible sheet of the present disclosure can be processed into a thin form such as a sheet, has excellent adhesiveness to the mucous membrane in the oral cavity, has excellent durability, and is decomposed at an appropriate rate by moisture in the oral cavity, and can be used for various purposes.

### BRIEF DESCRIPTION OF DRAWINGS

FIG. 1 is a graph illustrating the durability evaluation results according to the zinc content of an edible sheet according to an embodiment of the present disclosure.

FIG. 2 is a graph illustrating the evaluation result of the adhesion force of the edible sheet according to an embodiment of the present disclosure.

FIG. 3 is a graph illustrating the durability evaluation results of the edible sheet according to an embodiment of the present disclosure.

FIGS. 4 and 5 are graphs illustrating the evaluation results of the degradability of the edible sheet according to an embodiment of the present disclosure, respectively.

FIGS. 6 and 7 are graphs illustrating the metal ion dissolution evaluation results of the edible sheet according to an embodiment of the present disclosure, respectively.

### DETAILED DESCRIPTION OF THE INVENTION

Hereinafter, the function and the effect of the invention are presented in more detail through specific examples of the invention. However, the following examples are only for illustrating the invention and the scope of the invention is not limited to or by them.

### EXAMPLES

The reagents used are as follows.

Hyaluronic Acid: Manufacturer: BLOOMAGE BIOTECHNOLOGY CORP, Product Name: Hyaluronic Acid (HA-F-016)

Hypromellose: Manufacturer: ES R&D Center, Product name: Hydroxypropylmethylcellulose

Glycerin: Manufacturer: ES R&D Center, Product Name: Glycerin (Chemical Synthetic Food Additive)

Catechin: Manufacturer: Healing Co., Ltd. Product name: Catechin 24

L-Menthol: Manufacturer: Tien Yuan Chemical (PTE) LTD. Product Name: L-Menthol

Zinc Sulfate: Manufacturer: Serim Food Ingredients, Product Name: Zinc Sulfate

Preparation of Sheet (Patch) Type Food

### Example 1-1 (without Zinc)

About 500 g of purified water was prepared and heated to about 100° C. 20 g of hypromellose and 2 g of a hyaluronic acid were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-D084), and dried at about 100° C. for about 30 minutes to obtain a sheet (patch) type food.

The dry thickness was found to be about 0.3 mm.

### Example 1-2

About 500 g of purified water was prepared, heated to about 100° C., and about 0.58 g of zinc sulfate was added thereto, and then completely dissolved by stirring.

20 g of hypromellose and 2 g of a hyaluronic acid were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-D084), and dried at about 100° C. for about 30 minutes to obtain a sheet (patch) type food.

The dry thickness was found to be about 0.3 mm.

### Example 1-3

About 500 g of purified water was prepared, heated to about 100° C., and about 5.76 g of zinc sulfate was added thereto, and then completely dissolved by stirring.

20 g of hypromellose and 2 g of a hyaluronic acid were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-D084), and dried at about 100° C. for about 30 minutes to obtain a sheet (patch) type food.

The dry thickness was found to be about 0.3 mm.

### Example 1-4

About 500 g of purified water was prepared, heated to about 100° C., and about 57.6 g of zinc sulfate was added thereto, and then completely dissolved by stirring.

20 g of hypromellose and 2 g of a hyaluronic acid were added and dispersed through stirring.

The above dispersion was placed in a stainless tray, closed, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-D084), and dried at about 100° C. for about 30 minutes to obtain a sheet (patch) type food.

The dry thickness was found to be about 0.3 mm.

Durability Evaluation

The edible sheet prepared in the above examples was cut into a rectangular shape of 5 cm in width and 0.5 cm in length to prepare a specimen.

About 0.5 cm of the central portion of the specimen was lightly wetted with distilled water, and then the water was wiped off.

Tensile strength was measured using a physical property analyzer (Yeonjin S-Tech, TXA texture analyzer).

Measurements were repeated 5 times in the same manner to obtain an average value.

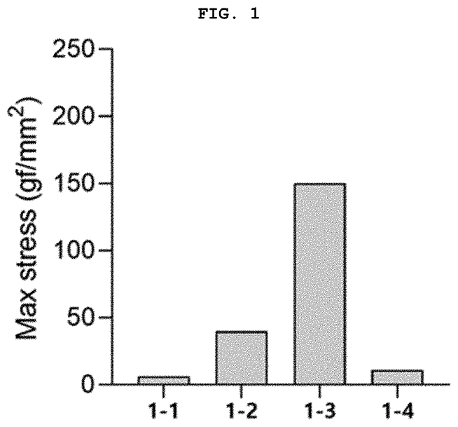

The measurement results are summarized in FIG. 1 below.

FIG. 1 is a graph illustrating the durability evaluation results according to the zinc content of an edible sheet according to an embodiment of the present disclosure.

Referring to FIG. 1, it was clearly identified that the tensile strength value increased from a minimum of two times to a maximum of several tens of times as zinc ions are added. In particular, in the case of Examples 1-3, it was clearly identified that the strength was significantly improved compared to other examples in which zinc ions are added.

As discussed above, this improvement in tensile strength is thought to be due to electrostatic interactions with metal ions and numerous hydroxy groups present in hyaluronic acid and hypromellose.

### Example 2-1 (Addition of Zinc and Other Additives)

250 g of purified water, 3.28 g of xylitol and 0.75 g of acesulfame potassium were added, and completely dissolved at room temperature (first liquid).

Separately, 250 g of purified water was heated to about 100° C., and 24 g of glycerin, 1 g of catechin, 3 g of L-menthol, and 11.52 g of zinc sulfate were added thereto, and then completely dissolved by stirring (second Liquid).

20 g of hypromellose and 2 g of a hyaluronic acid were added to the second solution, and dispersed through stirring.

The first solution and the second solution were mixed, placed in a stainless tray, closed with a lid, and left at room temperature for about 3 days. When water droplets formed on the lid were identified while standing, the lid was opened and the water formed on the lid was removed by wiping. After standing for three days, a sol-gel intermediate fluid with high viscosity was obtained.

Using an applicator, the fluid was uniformly applied to a thickness of about 1.5 mm on a release film (Skyrol SKC Polyester Film. SG00), transferred to a dryer (CORETECH, HQ-D084), and dried at about 100° C. for about 30 minutes to obtain a sheet (patch) type food.

The dry thickness was found to be about 0.3 mm.

### Examples and Comparative Examples

In Example 2-1, a sheet (patch) type food was prepared in the same manner as in Example 2-1, except that the contents of hypromellose and hyaluronic acid were different.

The content is summarized in Table 1 below.

TABLE 1

| | Hypromellose (g) | hyaluronic acid (g) |
|---|---|---|
| Example 2-1 | 21 | 1 |
| Example 2-2 | 20 | 2 |
| Example 2-3 | 19 | 3 |
| Example 2-4 | 18 | 4 |
| Example 2-5 | 17 | 5 |
| Example 2-6 | 16 | 6 |
| Example 2-7 | 15 | 7 |
| Example 2-8 | 14 | 8 |

Adhesion Force Evaluation

For the sheet (patch) type foods prepared in the examples and comparative Examples, the adhesion force in the oral cavity was evaluated by the following method.

First, in order to create an experimental environment similar to the soft tissue in the oral cavity in terms of adhesion force, subcutaneous tissue of a pig was prepared.

Pig subcutaneous tissue was cut into a rectangular shape with a width of 1.5 cm and a length of 10 cm, and two pieces thereof were prepared.

The fat portion of the pig subcutaneous tissue was scraped with a blade to remove the fat layer, and the scraped surface was wiped with 80 wt % ethanol.

The edible sheet prepared in the Examples and Comparative Examples was cut to the same size as the pig subcutaneous tissue, lightly wetted with distilled water, placed between the two pieces of pig subcutaneous tissue, and then the two pieces of pig subcutaneous tissue were overlapped without applying any pressure other than gravity, and placed on the floor and joined.

Using a physical property analyzer (Yeonjin S-Tech, TXA texture analyzer), the maximum load applied at this time was measured while pulling the two overlapping pig subcutaneous tissues to both sides and removing the same.

Measurements were repeated 5 times in the same manner to obtain an average value.

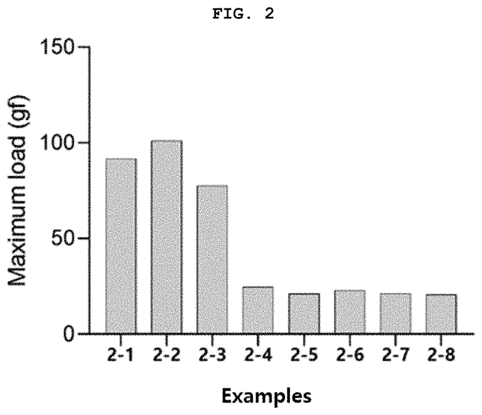

The measurement results are summarized in FIG. 2 below.

FIG. 2 is a graph illustrating the evaluation result of the adhesion force of the edible sheet according to an embodiment of the present disclosure.

Referring to FIG. 2, it was identified that the edible sheet according to an embodiment of the present disclosure has very good adhesion force to the human body. In particular, in the case of Examples 2-1 to 2-3, in which the content of a hyaluronic acid is relatively low, the maximum load required for separation from the pig subcutaneous tissue is about twice or more, compared to other examples, indicating that the adhesion strength was very good.

Durability Evaluation

The edible sheet prepared in the above examples and comparative examples was cut into a rectangular shape of 5 cm in width and 0.5 cm in length to prepare a specimen.

About 0.5 cm of the central portion of the specimen was lightly wetted with distilled water, and then the water was wiped off.

Using a physical property analyzer (Yeonjin S-Tech, TXA texture analyzer), the stress-strain was measured until the specimen broke while pulling both sides of the specimen, and the breaking strength thereof was measured.

Measurements were repeated 5 times in the same manner to obtain an average value.

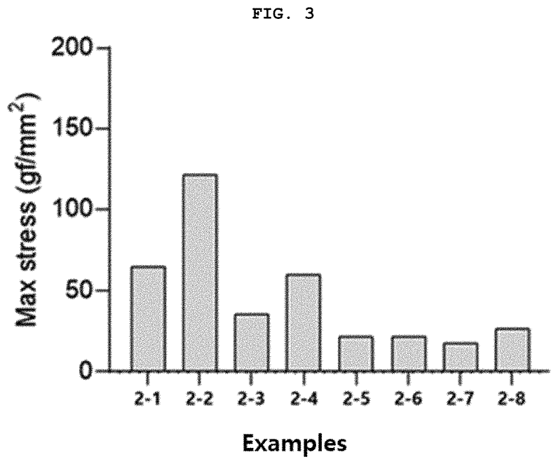

The measurement results are summarized in FIG. 3 below.

FIG. 3 is a graph illustrating the durability evaluation results of the edible sheet according to an embodiment of the present disclosure.

Referring to FIG. 3, it was identified that the edible sheet according to an embodiment of the present disclosure was not easily broken even after being wetted with water because of the high breaking strength thereof. In particular, in the case of Examples 2-1 to 2-4, in which the content of a hyaluronic acid is relatively small, the breaking strength value was very high compared to other examples, indicating that the durability thereof was very good.

Degradability Evaluation

The edible sheet prepared in the examples and comparative examples was cut into a rectangular shape of 1.5 cm in width and 1 cm in length to prepare three specimens.

The specimen and 3 ml of phosphate buffer were placed in a conical tube, the lid was closed, and then stored in an incubator at 37° C.

After 30 minutes, 1 hour, 2 hours, 4 hours, 8 hours, 16 hours, 32 hours, 64 hours, and 96 hours, 1 ml of the solution in each conical tube was extracted and stored in a petri dish, and was again supplemented with 1 ml of phosphate buffer in a conical tube.

The petri dish was placed into a dryer, dried, and the weight was measured, and the weight of the patch dissolved in the phosphate buffer aqueous solution was measured.

Measurements were repeated 5 times in the same manner to obtain an average value.

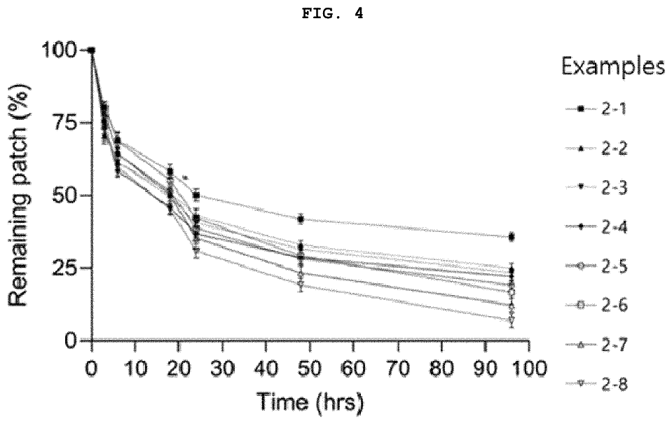

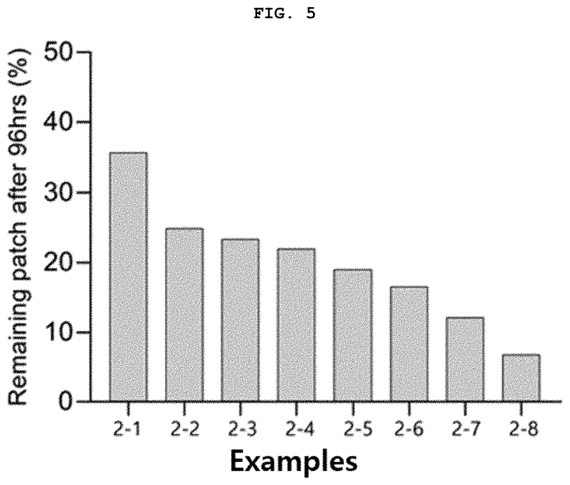

The measurement results are summarized in FIGS. 4 and 5 below.

FIGS. 4 and 5 are graphs illustrating the evaluation results of the degradability of the edible sheet according to an embodiment of the present disclosure, respectively.

Referring to FIG. 4, it was identified that it was most uniformly dissolved when the HPMC:HA ratio was 21:1. Referring to FIG. 5, it was identified that when the HPMC:HA ratio was 21:1, the largest amount remained, even after 96 hours, and the higher the HA ratio, the faster the erosion occurred.

Zinc Dissolution Evaluation

The edible sheet prepared in the examples and comparative examples was cut into a rectangular shape of 5 cm in width and 0.5 cm in length to prepare a specimen.

The specimen and 3 ml of phosphate buffer were placed in a conical tube, the lid was closed, and then stored in an incubator at 37° C. (the experiment was carried out by preparing 10 identical specimen each.).

Samples were taken out one by one over time, and the concentration of eluted zinc in the specimen was measured.

Measurements were repeated 5 times in the same manner to obtain an average value.

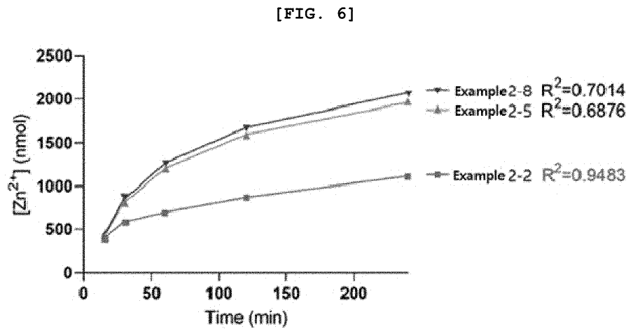

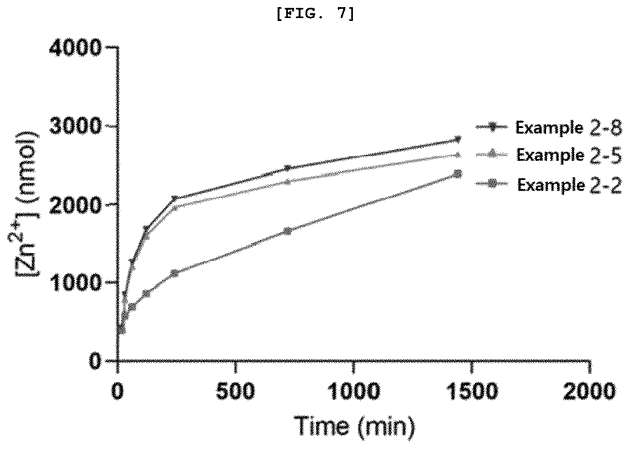

The measurement results are summarized in FIGS. 6 and 7 below.

FIGS. 6 and 7 are graphs illustrating the metal ion dissolution evaluation results of the edible sheet according to an embodiment of the present disclosure, respectively.

Referring to FIGS. 6 and 7, it was clearly identified that in the edible sheet according to an embodiment of the present disclosure, the release rate of the other components included in the sheet may be controlled according to the ratio of the hydrogel during the preparation of the sheet.

In particular, in the case of Example 2-2, the R² value is about 0.95, which is very close to 1, and it was identified that the components included in the sheet are released almost uniformly according to the time proportion under conditions similar to those of the human body.

Accordingly, it is considered that the edible sheet according to an embodiment of the present disclosure can be used not only as a simple food, but also as a health functional food containing a specific active ingredient, a patch for drug release, and the like.

1. An edible sheet comprising hyaluronic acid and hypromellose.

2. The edible sheet according to claim 1, wherein the hyaluronic acid comprises one or more of a cross-linked hyaluronic acid and a non-cross-linked hyaluronic acid.

3. The edible sheet according to claim 1, wherein the hypromellose has a methoxy substitution rate of 10 to 35%, and a hydroxypropyl substitution rate of 1 to 15%.

4. The edible sheet according to claim 1, wherein the edible sheet has a dry thickness of 0.05 to 1 mm.

5. The edible sheet according to claim 1, wherein the edible sheet includes 0.1 to 20 parts by weight of a hyaluronic acid, based on 100 parts by weight of hypromellose.

6. The edible sheet according to claim 1, wherein the edible sheet includes 0.1 to 18 parts by weight of a hyaluronic acid and 82 to 99.9 parts by weight of hypromellose, based on a total of 100 parts by weight of a hyaluronic acid and hypromellose.

7. The edible sheet according to claim 1, wherein the hyaluronic acid has a weight average molecular weight of 10,000 to 1,000,000 g/mol.

8. The edible sheet according to claim 1, wherein the hypromellose has a weight average molecular weight of 100,000 to 1,000,000 g/mol.

9. The edible sheet according to claim 1, further including ions of one or more edible metals selected from the group consisting of zinc, copper, iron, nickel, manganese, chromium, calcium, magnesium, sodium, potassium and selenium.

10. The edible sheet according to claim 9, wherein the hyaluronic acid and the hypromellose are in the form of a complex coordinated around the metal.

11. The edible sheet according to claim 9, wherein the ion of the metal is included in a ratio of 0.01 to 2 moles with respect to a total of 100 g of the hyaluronic acid and the Hypromellose.
